(19) United States
(12) Patent Application Publication
(10) Pub. No.: US 2013/0131493 A1
(43) Pub. Date: May 23, 2013

(54) METHOD AND APPARATUS FOR PERFORMING DUAL-MODALITY IMAGING

(71) Applicants: Scott David Wollenweber, Waukesha, WI (US); Alexander Ganin, Whitefish Bay, WI (US); Joan M. Hanson, Cambridge, WI (US); William J. Bridge, Delafield, WI (US); Kathryn M. Littlejohn, Wales, WI (US); Ling Sun, Brookfield, WI (US)

(72) Inventors: Scott David Wollenweber, Waukesha, WI (US); Alexander Ganin, Whitefish Bay, WI (US); Joan M. Hanson, Cambridge, WI (US); William J. Bridge, Delafield, WI (US); Kathryn M. Littlejohn, Wales, WI (US); Ling Sun, Brookfield, WI (US)

(73) Assignee: GENERAL ELECTRIC COMPANY, Schenectady, NY (US)

(21) Appl. No.: 13/302,701

(22) Filed: Nov. 22, 2011

(51) Int. Cl. A61B 5/055; A61B 6/00

(52) U.S. Cl. 600/411

(57) ABSTRACT

A method for performing an examination of a patient using a dual-modality imaging system includes accessing a list that includes a magnetic resonance imaging (MRI) examination protocol to be used to perform a predetermined MRI examination, the examination protocol including a plurality of MRI protocol actions, receiving an input selecting at least one of the MRI protocol actions, scanning the patient using the MRI examination protocol, scanning the patient using a positron emission tomography (PET) imaging system to acquire PET data, and marking the PET data, using a first visual indication, to indicate the selected MRI protocol action. An imaging system and a non-transitory computer readable medium are also described herein.

### BACKGROUND OF THE INVENTION

The subject matter disclosed herein relates generally to diagnostic imaging systems, and more particularly to a method and apparatus for performing a medical examination of a patient using a dual-modality imaging system.

Positron emission tomography (PET) imaging systems typically generate images depicting the distribution of positron-emitting nuclides in patients. The positron interacts with an electron in the body of the patient by annihilation, with the electron-positron pair converted into two photons. The photons are emitted in opposite directions along a line of response. The annihilation photons are detected by detectors (that are typically in a detector ring assembly) on both sides of the line of response on the detector ring assembly. These detections are termed coincidence events. The coincidence events detected by the PET detector ring assembly are typically stored within data structures called emission sinograms, which is a histogram of the detected coincidence events. An image of the activity distribution within a patient's body is generated from the emission sinograms through a process called image reconstruction.

A magnetic resonance (MR) imaging system is a medical imaging modality that generates images of the inside of a human body without using x-rays or other ionizing radiation. MR systems typically include a powerful magnet to create a strong, uniform, static magnetic field (i.e., the “main magnetic field”). When a human body, or part of a human body, is placed in the main magnetic field, the nuclear spins that are associated with the hydrogen nuclei in tissue water become polarized. This means that the magnetic moments that are associated with these spins become preferentially aligned along the direction of the main magnetic field, resulting in a small net tissue magnetization along an imaging axis, typically the z-axis. An MRI system also includes called gradient coils that produce smaller amplitude, spatially varying magnetic fields when current is applied. Typically, gradient coils are designed to produce a magnetic field component that is aligned along the z axis, and that varies linearly in amplitude with position along one of the x, y or z axes. The effect of a gradient coil is to create a small ramp on the magnetic field strength, and concomitantly on the resonant frequency of the nuclear spins, along a single axis.

A dual-modality imaging system may include both the PET imaging system and the MR imaging system. However, at least some techniques utilized to acquire the MR information, such as for example, applying gradient pulses using the gradient coils, may cause a reduction in the quality of the information acquired using the PET imaging system. More specifically, using various MR imaging techniques may interfere with the collection of PET data thus reducing the quality of the PET data collected during the examination. As a result, the quality of reconstructed images may also be reduced.

### BRIEF DESCRIPTION OF THE INVENTION

In one embodiment, a method for performing an examination of a patient using a dual-modality imaging system is provided. The method includes accessing a list that includes a magnetic resonance imaging (MRI) examination protocol to be used to perform a predetermined MRI examination, the examination protocol including a plurality of MRI protocol actions, receiving an input selecting at least one of the MRI protocol actions, scanning the patient using the MRI examination protocol, scanning the patient using a positron emission tomography (PET) imaging system to acquire PET data, and marking the PET data, using a first visual indication, to indicate the selected MRI protocol action.

In another embodiment, a dual-modality imaging system is provided. The dual-modality imaging system includes a magnetic resonance imaging (MRI) system, a positron emission tomography (PET) imaging system, and a computer coupled to the MRI system and the PET system. The computer is configured to access a list that includes a magnetic resonance imaging (MRI) examination protocol to be used to perform a predetermined MRI examination, the examination protocol including a plurality of MRI protocol actions, receive a user input selecting at least one of the MRI protocol actions, scan the patient using the MRI examination protocol, scan the patient using a positron emission tomography (PET) imaging system to acquire PET data, and mark the PET data to indicate the selected MRI protocol action.

In a further embodiment, a non-transitory computer readable medium is provided. The computer readable medium is programmed to instruct a computer to access a list that includes a magnetic resonance imaging (MRI) examination protocol to be used to perform a predetermined MRI examination, the examination protocol including a plurality of MRI protocol actions, receive a user input selecting at least one of the MRI protocol actions, scan the patient using the MRI examination protocol, scan the patient using a positron emission tomography (PET) imaging system to acquire PET data, and mark the PET data to indicate the selected MRI protocol action.

### BRIEF DESCRIPTION OF THE DRAWINGS

FIG. 1 is a flowchart of an exemplary method of imaging a patient in accordance with various embodiments.

FIG. 2 is an exemplary list that may be generated in accordance with various embodiments.

FIG. 3 is another exemplary list that may be generated in accordance with various embodiments.

FIG. 4 is an exemplary image that may be generated in accordance with various embodiments.

FIG. 5 is another exemplary image that may be generated in accordance with various embodiments.

FIG. 6 is an exemplary block diagram illustrating a portion of the method described herein in accordance with various embodiments.

FIG. 7 is perspective view of a multi-modality imaging system formed in accordance with various embodiments.

FIG. 8 is a block diagram of a portion of the exemplary imaging system shown in FIG. 7 in accordance with various embodiments.

FIG. 9 is a block diagram of another portion of the exemplary imaging system shown in FIG. 7 in accordance with various embodiments.

### DETAILED DESCRIPTION OF THE INVENTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Described herein are various embodiments for performing an examination of a patient using a dual-modality imaging system. Various embodiments enable magnetic resonance (MR) examination procedure markers to be added to, or overlaid onto, positron emission tomography (PET) data. At least one known technical effect of various embodiments is to enable the user to determine which PET data are most useful based on the examination procedure utilized to acquire the PET data. More specifically, the markers enable the PET data to be retrospectively sorted based upon the MRI examination be utilized.

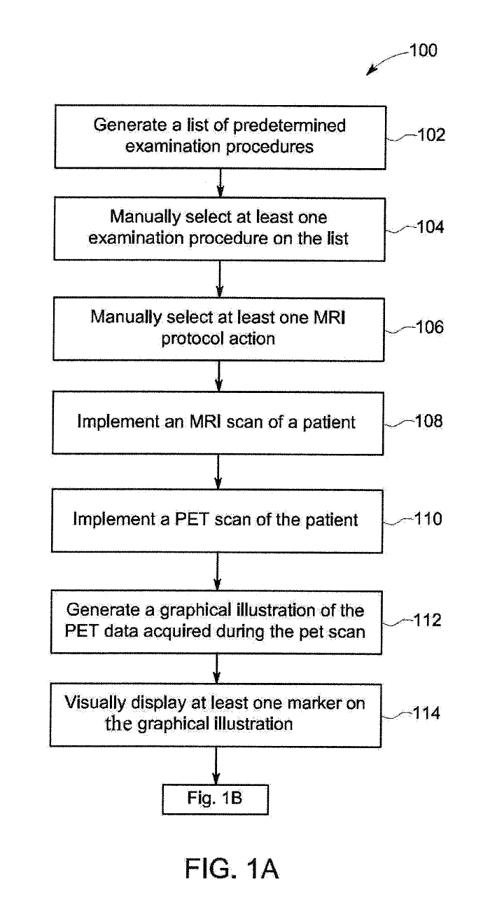

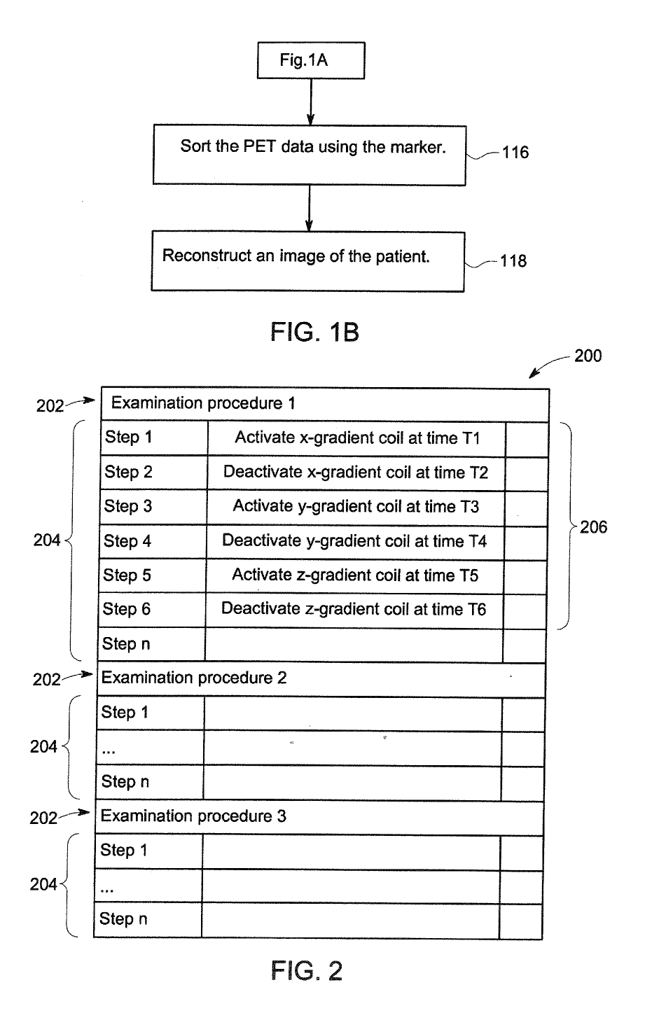

FIG. 1 is a flowchart of an exemplary method 100 for performing an examination of a patient using a dual-modality imaging system. The method 100 may be stored as a list of instructions, for example, on a computer coupled to the dual-modality imaging system. In various embodiments, the method 100 is described with respect to a dual-modality imaging system that includes a MRI system and a PET imaging system. It should be realized that other modalities may be utilized with the dual-modality imaging system. Other modalities include, for example, a computed tomography (CT) imaging system and a single photon emission computed tomography (SPECT) imaging system.

At 102, a list of predetermined MRI examinations is generated. Exemplary MRI examination 202 may include, for example, brain imaging, spine imaging, and/or performing musculoskeletal imaging, etc. For example, FIG. 2 illustrates an exemplary list 200 of exemplary MRI examination 202 that may be generated at 102. In various embodiments, the list 200 includes a plurality of MRI examinations 202 (illustrated as examination procedures) that may be performed using an MRI imaging system. The examinations 202 are each defined by a scan protocol 204. The scan protocol 204 is a set of procedural steps that are implemented by the MRI imaging system to conduct the MRI examination 202.

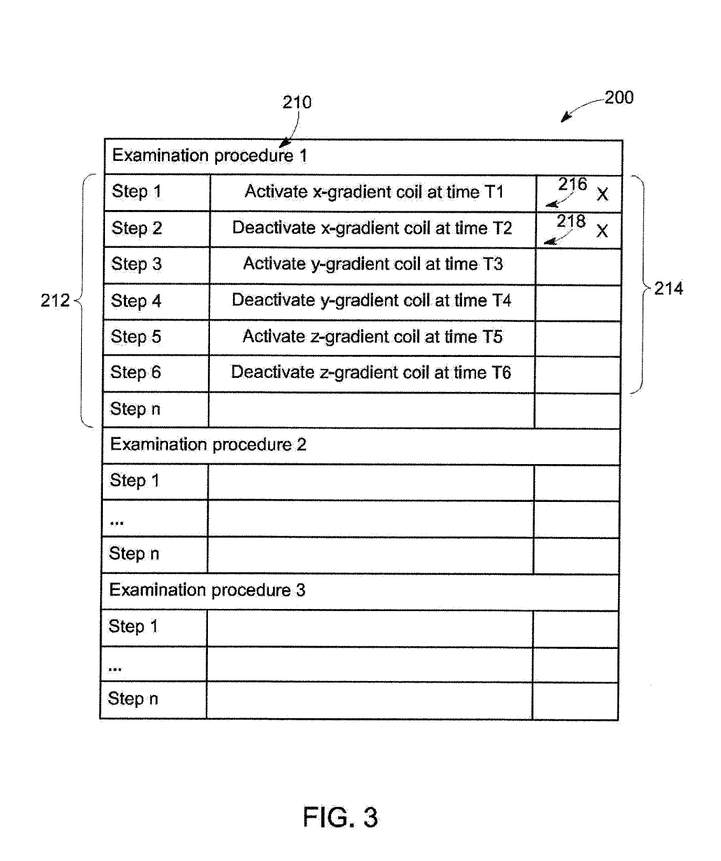

The scan protocol 204 is, in the exemplary embodiment, generated based on a priori information of the MRI examination procedure 202. More specifically, as discussed above, exemplary MRI examinations 202 may include, for example, brain imaging, spine imaging, and/or performing musculoskeletal imaging, etc. In the exemplary embodiment, the scan protocol 204 for a respective examination 202 is configured to operate the MRI imaging system in such a manner as to generate the information requested by the user for the associated medical examination. The scan protocol 204 may be implemented as a set of instructions to either activate or deactivate various components of the MRI imaging system in a predetermined manner to acquire the requested MRI data. For example, as shown in FIG. 3, a scan protocol 206 for a first examination procedure 208, such as an MRI brain scan, may include instructions to activate, for example, an x-gradient coil at time T1, to deactivate the x-gradient coil at time T2, to activate a y-gradient coil at time T3, to deactivate the y-gradient coil at a time T4, to activate a z-gradient coil at a time T5, and/or to deactivate the z-gradient coil at a time T6. It should be realized that the above described MRI imaging system components, and the operation thereof, are exemplary only, and that various other MRI imaging system components may be operated based on the selected MRI scan protocol.

It should be realized that the list of steps forming the scan protocols 204 shown in FIG. 2 is exemplary, and that various steps may be added to, or deleted from the examination procedures, based on revisions to the examination desired to be performed of the patient (e.g. modifications by the user). It should be further realized that the list 200, may include a plurality of scan protocols and associated steps utilized to carry out the associated scan protocols for a plurality of different examination procedures. For example, the list 200, shown in FIG. 2, may include for example, an examination procedure for brain imaging, an examination procedure for spine imaging, an examination procedure for musculoskeletal imaging, etc. Additionally, each examination procedure includes an associated list of steps, referred to herein as a scan protocol, to perform the examination procedure.

In the exemplary embodiment, a scan protocol 204 is a list of actions 206 that represent a sequence of operational steps instructing the MRI imaging system to perform some action at some predetermined time. More specifically, the scan protocol 204 for a selected examination 202 is configured to operate the MRI imaging system in a desired manner to achieve the information desired by the user. Thus, in the exemplary embodiment, the scan protocol 204 includes a list of steps 206, also referred to herein as MRI scan protocol actions 206, that instruct the MRI imaging system to operate the MRI system in some predetermined manner to enable the MRI system to acquire the MRI information requested by the user. In various embodiments, the user may manually alter the scan protocol 204 by adding or deleting scan protocol actions 206 to the scan protocol 204. For example, the user may manually modify the examination protocol shown in FIG. 2 or 3 by manually changing various system inputs while the MRI system is scanning the patient to reconfigure the MRI system in various different operational configurations.

Referring again to FIG. 1, at 104, a user manually selects at least one of the examination procedures 202. The user may manually select the examination procedure 202 by, for example, utilizing a manual user interface to select one of the examinations 202 shown on FIG. 2. Optionally, the user may select an icon on a display associated with the desired examination 202. For example, the user may manually select the examination procedure 210 shown in FIG. 3. The examination procedure 210 includes a scan protocol 212 having a plurality of scan protocol actions 214.

At 106, the user manually selects at least one of the MRI protocol actions 214 listed in the respective examination procedure. For example, as shown in FIG. 3, the user may select the scan protocol actions 216 and 218. The user may select the scan protocol actions 216 and 218 by, for example, utilizing the user interface to place a check or some other mark next to the selected scan protocol actions 216 and 218. As discussed above, the scan protocol actions 214 form a list of actions to be performed by the MRI imaging system to implement the examination 200. Accordingly, at 106 the user may identify at least one of the scan protocol actions 214 that the user desires to monitor and utilize for image reconstruction as discussed in more detail below.

At 108, the MRI system implements the selected examination procedure. For example, the MRI imaging system may implement the selected examination procedure 210 as discussed above. In operation, the MRI imaging system then implements the scan protocol 212 by performing the individual scan protocol actions 214 as shown in FIG. 3. For example, at time T1, the MRI imaging system activates the x-gradient coil, at time T2, the MRI imaging system deactivates the x-gradient coil, at time T3, the MRI imaging system activates the y-gradient coil, at time T4, the MRI imaging system deactivates the y-gradient coil, at a time T5, the MRI imaging system activates the z-gradient coil, and at a time T6, the MRI imaging system deactivates the z-gradient coil. After the scan protocol 212 is completed the examination 210 is completed.

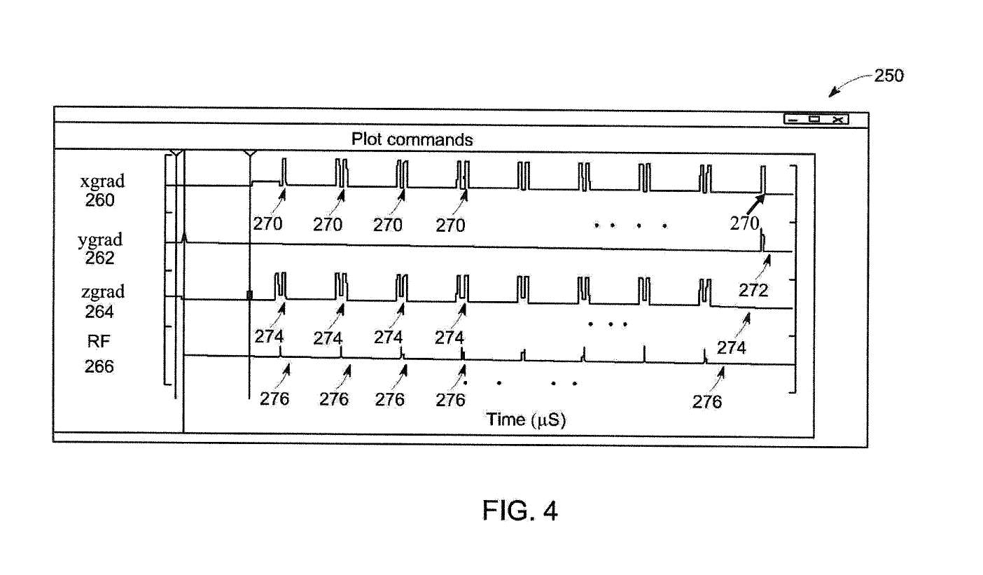

The information acquired from the examination 210 is stored in a computer for later use as described in more detail below. Moreover, the MRI scan protocol 212, including the individual scan protocol actions 214 may be utilized to generate a graphical illustration of the examination procedure 210. For example, FIG. 4 is a graphical illustration 250 denoting the various scan protocol actions 214 utilized to implement the examination procedure 210. More specifically, FIG. 4 illustrates the operation of an x-gradient coil 260, a y-gradient coil 262, a z-gradient coil 264, and an RF coil 266. As shown in FIG. 4, the x-gradient coil 260 may be activated and deactivated at various times during the examination procedure 210 as denoted by arrows 270. The y-gradient coil 266 may be activated and deactivated at various times during the examination procedure 210 as denoted by arrows 272, the z-gradient coil 264 may be activated and deactivated at various times during the examination procedure 210 as denoted by arrows 274, and the RF coil 266 may be activated and deactivated at various times during the examination procedure 210 as denoted by arrows 276.

Referring again to FIG. 1, at 110, the PET imaging system implements a selected examination procedure. The PET examination procedure may be any procedure to acquire physiological information of the patient. In various embodiments, the PET examination may be implemented using an imaging agent. The term “imaging agent,” as used herein includes any and all radiopharmaceutical (RP) agents and contrast agents used in connection with diagnostic imaging and/or therapeutic procedures. The imaging agent may represent a perfusion agent. The imaging agent may be, among other things, an imaging agent adapted for use in MRI or functional MRI, an intravenous CT contrast agent, a radiopharmaceutical PET or a SPECT tracer, an ultrasound contrast agent, an optical contrast agent, myocardial perfusion tracers, cerebral perfusion tracer and the like. By way of example only, the imaging agent may be Myoview™, Fluorodeoxyglucose (FDG), ¹⁸F-Flourobenzyl Triphenyl Phosphonium (¹⁸F-FBnTP), ¹⁸F-Flouroacetate, ¹⁸F-labled myocardial perfusion tracers, Tc-ECD, Tc-HMPAO, N-13 ammonia, Envision N-13H3, Iodine-123 ligands, ^{99m}-Technitium ligands, Xenon-133, Neuroreceptor ligands, etc.), 18F-fluoromisonidazole, ²⁰¹Thallium, ^{99m}Technetium sestamibi, and ⁸²Rubidium among others.

In various embodiments, the imaging agent is selected based on the particular type of PET examination desired. For example, the FDG agent may be utilized during a brain study. The various embodiments may be utilized by a variety of imaging modalities to provide study specific reference regions that may vary in terms of size, shape and/or location in the body. In the exemplary embodiment, the PET examination is performed on the same region of interest as the MRI examination. Thus, the imaging agent utilized to perform the PET examination may be selected based on both the PET examination desired and the MRI examination desired.

In the exemplary embodiment, the PET examination procedure is implemented substantially concurrently with the MRI examination performed at 108. For example, in operation, the patient may be positioned on a table. The table, including the patient, may then be moved within a gantry of the imaging system. The MRI imaging system may then be activated to acquire MRI imaging data of the patient, using for example, the examination procedure selected at 104. Additionally, while the MRI examination is being performed, the PET examination is being performed. Thus MRI imaging data is acquired substantially concurrently while the PET emission data is acquired.

The PET emission dataset may be stored in any format, such as a list mode dataset, for example. The PET emission dataset may be obtained during real-time scanning of the patient, as described above. For example, the methods described herein may be performed on emission data as the emission data is received from the PET imaging system during a real-time examination of the patient.

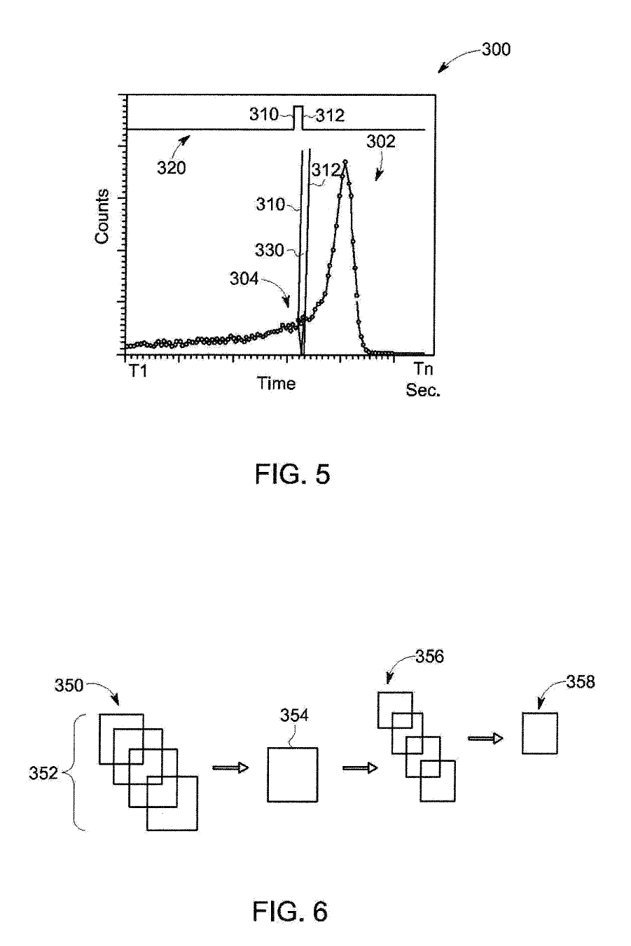

At 112, a graphical image of the PET data acquired at 110 is generated. For example, FIG. 5 illustrates an exemplary graphical image 300 that may be generated using PET emission data 302 acquired at 112. In the exemplary embodiment, the image 300 is the counts in a sinogram slice through the patient wherein the x-axis denotes the acquisition time and the y-axis denotes counts. It should be realized that although counts in a single sinogram are shown, that in operation, the methods described herein are configured to generate, and display data from a plurality of sinograms of the patient being imaged. Moreover, the sinograms of each slice may be displayed individually or may be collated and displayed as single sinogram. Moreover, it should be realized that the PET emission data acquired at 110 may be displayed in a variety of manners known in the art. For example, the emission data may be displayed as a histogram.

Referring again to FIG. 1, at 114, at least one marker identifying at least one of the protocol actions selected at 106 is displayed on the image 300. As discussed above, various MR imaging techniques may interfere with the collection of PET data thus reducing the quality of the PET data collected during the examination. For example, and referring again to FIG. 5, the emission dataset 302 includes at least one unexplained data anomaly 304 which is represented as a sharp drop in counts from a normal expected count rate. As a result, an image reconstructed using the reduced quality data shown in FIG. 5, may also have a reduced quality.

Accordingly, in the exemplary embodiment, at 106 the scan protocol actions 216 and 218 were selected. More specifically, the user indicated that information indicating that the x-gradient coil was activated and deactivated were of importance. Accordingly, at 114 a pair of markers is displayed on the image 300. For example, a first marker 310 may indicate that the x-gradient coil was activated and a second different marker 312 may indicate that the x-gradient coil was deactivated. In various embodiments, the various markers, including the markers 310 and 312 may be displayed as vertical lines that are overlaid on the image 300. In various other embodiments, the markers, including the markers 310 and 312, may be displayed as a separate graph that is overlaid on the image 300. For example, the image 300 may include a graph 320 that shows the operation of the x-gradient coil during the MRI examination procedure 200. In the exemplary embodiment, the graph 320 may include the markers 310 and 312 that show the x-gradient coil being activated and deactivated, respectively.

As shown in FIG. 5, an area 330, which is defined between the markers 310 and 312, may include invalid or non-optimal emission data. More specifically, in the exemplary embodiment, the operation of the x-gradient coil may have interfered with the acquisition of the PET emission data between time T1 and T2, which denote the operational times of the x-gradient coil. It should be realized that in various embodiments, the user may add or delete various markers to the image 300 to identify any various data anomalies indicated in the image 300.

Accordingly, and referring again to FIG. 1, at 116 the user may utilize the markers 310 and 312 to sort the PET emission data. For example, FIG. 6 illustrates an exemplary PET emission dataset 350 acquired during the PET examination procedure. The PET emission dataset 350 includes a plurality of sinograms 352 that each represent a particular slice of an image to be reconstructed. In operation, the user may delete the emission data in the area 330 (shown in FIG. 5), which is defined by the markers 310 and 312 to generate a second subsequent emission dataset 354 that includes the emission data acquired during the PET scan with the area 330 of emission data removed.

In various embodiments, the emission dataset 354 may be sorted into a plurality of bins 356. It should be realized that the quantity of bins illustrated in FIG. 6 is exemplary, and that during operation, fewer than four bins or more than four bins may be utilized. As such, each of the bins 356 includes approximately ¼ of the total information in the PET emission dataset 354.

For example, assume that the total length of the scan performed at 110 to acquire emission data is four minutes. Moreover, the imaging dataset 356 may be sorted into four bins, wherein each respective bin includes approximately one minute of information. In the exemplary embodiment, after the emission data is sorted into bins, the plurality of bins 356 may be utilized to reconstruct an image of the patient.

In another exemplary embodiment, the emission dataset 352 may be weighted to account for the data in the area 300. For example, assume that the data defined within the area 300 is considered to be invalid data and the emission data defined outside the area 300 is considered to be valid data. At 118, a fractional weight Wt may be assigned to the subset of invalid data, e.g. the data within the area 300. In one embodiment, the fractional weight Wt is calculated based on a fractional time that the PET imaging system was determined to be producing invalid data. For example, assuming that a duration of an exemplary scan is three minutes and assuming that during the scan the invalid data was produced for approximately 30 seconds, then the invalid data is removed from the PET emission dataset 350 to form the subset of valid image data 354 that has a duration of 150 seconds. Thus, during a three-minute scan, the PET imaging system is producing valid data for 150 seconds and the fractional weight Wt is calculated as 150/180=0.83. It should be realized that in the exemplary embodiment, list mode data is used to identify both the valid and invalid data and to calculate the fractional weight Wt. Thus, at 116 the entire emission dataset 350 may be sorted and utilized to reconstruct an image. Optionally, an invalid portion of the emission dataset may be deleted and the remaining portion, or valid emission data, may then be utilized to reconstruct an image of the patient.

At 118, the emission dataset 354 is utilized to reconstruct a PET image of the patient such as an image 358 shown in FIG. 6.

Described herein are various embodiments that enable analysis of concurrently collected PET and MRI data. Utilizing concurrently collected imaging data enables a user to determine which PET data is most useful for different purposes. For example, various methods enable the use of markers, to mark fMRI techniques, where simultaneous initiation of the fMRI stimulus is marked within the PET data, enabling retrospective sorting of the PET data based upon the fMRI scan status. Moreover, because some MRI techniques may interfere with the quality of the PET data, knowledge of the MRI technique being utilized to scan a patient may be used to sort or weight the PET data for the purpose of forming the highest quality PET images.

In operation, utilizing event markers within the PET data, enable retrospective PET data sorting and characterization. The markers enable the user to sort the PET data based on the presence or absence of events, in order to analyze the impact of different types of MRI events on the PET data. In various embodiments, the PET data may be marked to indicate various MRI events including, for example, RF pulse status, gradient coil status, etc., as well as external physiologic devices such as cardiac and respiratory devices. The event stream is shared simultaneously to the PET coincidence event acquisition system. The event markers may be stored within the temporal PET list of event data, e.g. listmode data. The listmode data may be sorted into sinograms, from which PET reconstruction subsequently forms images of activity concentration. Currently, PET listmode data is collected using time markers (e.g. 1 ms time markers) while the MRI system is capable of micro-second resolution.

A visual presentation of the event list may be displayed and the PET listmode data and sorted. In one embodiment, sorting may be accomplished in an interactive user-driven manner. In another embodiment, sorting may be accomplished in a configurable manner such that the user selects a protocol, such as ‘select only data starting at event marker X and lasting 30 seconds.’ The computer may then output a secondary set of PET listmode data that can subsequently be utilized within PET image formation. In a further embodiment, various methods may present a programmable capability to store a protocol for subsequent use and/or may be associated from disparate segments of the listmode data and stored as a secondary PET listmode dataset. Additionally, various embodiments enable the user to be notified in real-time of the PET data quality based on the current MR impact on PET, such as the user prescribing a 3-minute acquisition in a certain body location, but the real-time analysis of the event list suggesting that the user will only acquire a 2-minute equivalent of ‘quality’ PET data based on data analysis—and hence, another longer duration (‘scan extend’) could be suggested to the user. Additionally, the PET data may be weighted based on the quality of the PET data to increase the influence of the ‘valid’ PET data and decrease the influence of the potentially invalid PET data.

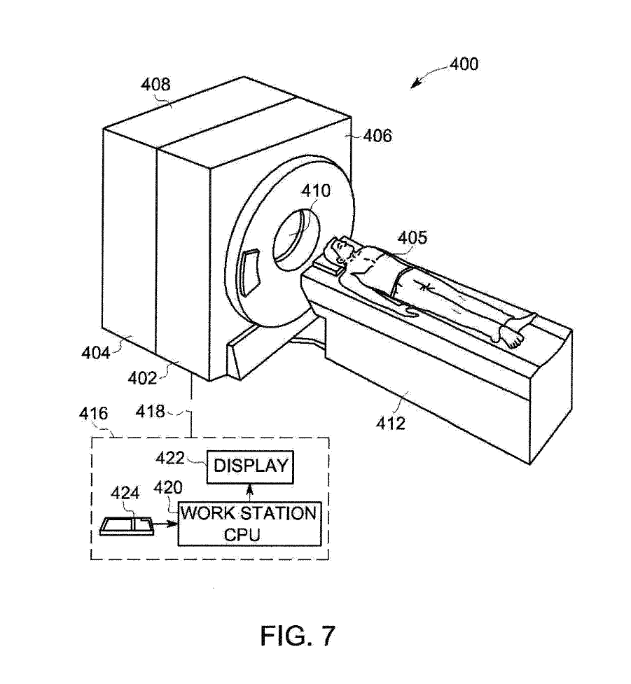

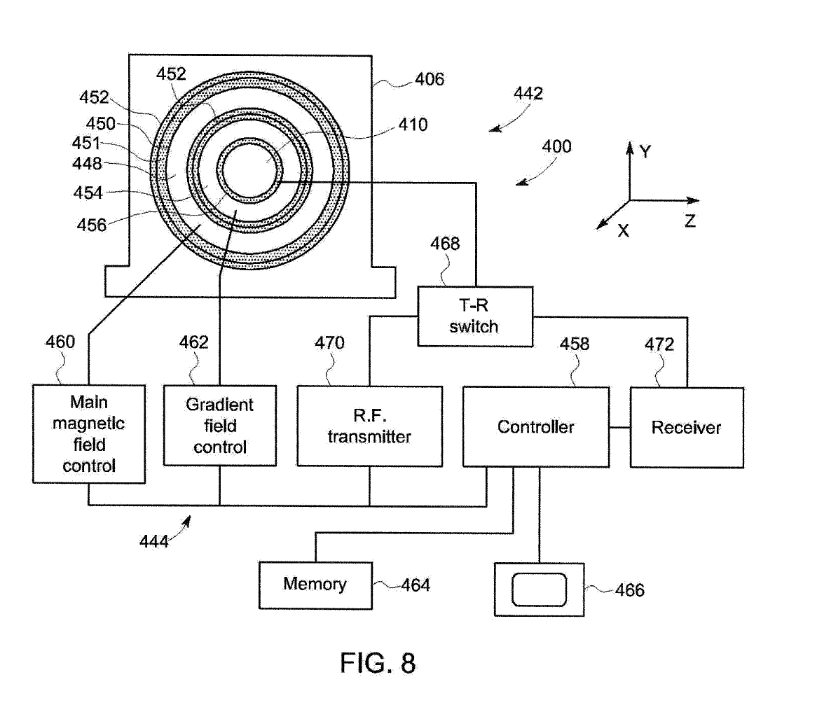

In various embodiments, the methods described herein may be implemented using a multi-modality imaging system. For example, FIGS. 7 and 8 illustrate embodiments of a multi-modality imaging system 400 in which one or more embodiments of the method 100 may be implemented. The multi-modality imaging system 100 may be any type imaging system, for example, different types of medical imaging systems, which in various embodiments, is an MRI system in combination with one of, for example, a PET system or a SPECT system capable of generating diagnostic images. Moreover, the various embodiments are not limited to medical imaging systems for imaging human subjects, but may include veterinary or non-medical systems for imaging non-human objects, etc., as well as non-imaging applications, such as radiation therapy.

Referring to FIG. 7, the multi-modality imaging system 400 includes a first modality unit 402 and a second modality unit 404. The two modality units enable the multi-modality imaging system 400 to scan an object or patient 405 in a first modality using the first modality unit 402, which in this embodiment is MR and to scan the patient 405 in a second modality using the second modality unit 404, which in this embodiment is PET. The multi-modality imaging system 400 allows for multiple scans in different modalities to facilitate an increased diagnostic capability over single modality systems. In one embodiment, the multi-modality imaging system 400 is an MR/PET imaging system and in another embodiment the multi-modality imaging system 400 is an MR/SPECT imaging system.

The imaging system 400 is shown as including a gantry 406 that is associated with the first modality unit 402, which is an MR scanner, and a gantry 408 that is associated with the second modality unit 404. During operation, the patient 405 is positioned within a central opening 410, defined through the imaging system 400, using, for example, a motorized table 412.

The gantry 406 includes MR imaging components, for example, one or more magnets as described in more detail herein. The gantry 408 includes imaging components for an X-ray radiation or gamma radiation type system (e.g., an x-ray tube and detector or gamma emission detectors or cameras, such as Cadmium Zinc Telluride (CZT) detector modules).

The imaging system 400 also includes an operator workstation 416. During operation, the motorized table 412 moves the patient 405 into the central opening 410 of the gantry 406 and/or 408 in response to one or more commands received from the operator workstation 416. The workstation 416 then operates the first and second modality units 402 and 404 to, for example, both scan the patient 405 in MR and acquire attenuation and/or emission data of the patient 405. The workstation 416 may be embodied as a personal computer (PC) that is positioned near the imaging system 400 and hard-wired to the imaging system 400 via a communication link 418. The workstation 416 may also be embodied as a portable computer such as a laptop computer or a hand-held computer that transmits information to, and receives information from, the imaging system 400. Optionally, the communication link 418 may be a wireless communication link that enables information to be transmitted to or from the workstation 416 to the imaging system 400 wirelessly. In operation, the workstation 416 is configured to control the operation of the imaging system 400 in real-time. The workstation 416 is also programmed to perform medical image diagnostic acquisition and reconstruction processes described herein.

The operator workstation 416 includes a central processing unit (CPU) or computer 420, a display 422, and an input device 424. In the exemplary embodiment, the computer 420 executes a set of instructions that are stored in one or more storage elements or memories, in order to process information received from the first and second modality units 402 and 404. For example, in various embodiments, the computer 416 may include a set of instructions to implement the method 100 described herein. The storage elements may also store data or other information as desired or needed. The storage element may be in the form of an information source or a physical memory element located within the computer 420. The set of instructions may include various commands that instruct the computer 420 as a processing machine to perform specific operations such as the methods and processes of the various embodiments described herein.

The computer 420 connects to the communication link 418 and receives inputs, e.g., user commands, from the input device 424. The input device 424 may be, for example, a keyboard, mouse, a touch-screen panel, and/or a voice recognition system, etc. Through the input device 424 and associated control panel switches, the operator can control the operation of the first and second modality units 402 and 404 and the positioning of the patient 405 for a scan. Similarly, the operator can control the display of the resulting image on the display 422.

Referring to FIG. 8, the imaging system 400 includes an imaging portion 442 and a processing portion 444 that may also include a processor or other computing or controller device, such as illustrated in FIG. 7. The imaging system 400 includes within a helium vessel 451 a superconducting magnet 448 formed from coils, which may be supported on a magnet coil support structure. The helium vessel 451 surrounds the superconducting magnet 448 and is filled with liquid helium.

Thermal insulation 452 is provided surrounding all or a portion of the outer surface of the helium vessel 451. A plurality of magnetic gradient coils 454, such as the x, y, and z gradient coils described above, are provided inside the superconducting magnet 448 and an RF transmit coil 456 is provided within the plurality of magnetic gradient coils 454. In some embodiments, the RF transmit coil 456 may be replaced with a transmit and receive coil. The components within the gantry 450 generally form the imaging portion 442. It should be noted that although the superconducting magnet 448 is a cylindrical shape, other shapes of magnets can be used.

The processing portion 444 generally includes a controller 458, a main magnetic field control 460, a gradient field control 462, a memory 465, a display device, embodied as the monitor 466, a transmit-receive (T-R) switch 468, an RF transmitter 470 and a receiver 472.

In operation, a body of an object, such as a patient or a phantom to be imaged, is placed in the bore 410 on a suitable support, for example, a patient table. The superconducting magnet 448 produces a uniform and static main magnetic field Bₒ across the bore 410. The strength of the electromagnetic field in the bore 410 and correspondingly in the patient, is controlled by the controller 458 via the main magnetic field control 460, which also controls a supply of energizing current to the superconducting magnet 448.

The magnetic gradient coils 454, which include one or more gradient coil elements, are provided so that a magnetic gradient can be imposed on the magnetic field Bₒ in the bore 410 within the superconducting magnet 448 in any one or more of three orthogonal directions x, y, and z. The magnetic gradient coils 454 are energized by the gradient field control 462 and are also controlled by the controller 458.

The RF transmit coil 456, which may include a plurality of coils, is arranged to transmit magnetic pulses and/or optionally simultaneously detect MR signals from the patient if receive coil elements are also provided, such as a surface coil configured as an RF receive coil. The RF receive coil may be of any type or configuration, for example, a separate receive surface coil. The receive surface coil may be an array of RF coils provided within the RF transmit coil 456.

The RF transmit coil 456 and the receive surface coil are selectably interconnected to one of the RF transmitter 470 or receiver 472, respectively, by the T-R switch 468. The RF transmitter 470 and T-R switch 468 are controlled by the controller 458 such that RF field pulses or signals are generated by the RF transmitter 470 and selectively applied to the patient for excitation of magnetic resonance in the patient. While the RF excitation pulses are being applied to the patient, the T-R switch 468 is also actuated to disconnect the receive surface coil from the receiver 472.

Following application of the RF pulses, the T-R switch 468 is again actuated to disconnect the RF transmit coil 456 from the RF transmitter 470 and to connect the receive surface coil to the receiver 472. The receive surface coil operates to detect or sense the MR signals resulting from the excited nuclei in the patient and communicates the MR signals to the receiver 472. These detected MR signals are in turn communicated to the controller 458. The controller 458 includes a processor (e.g., image reconstruction processor), for example, that controls the processing of the MR signals to produce signals representative of an image of the patient.

The processed signals representative of the image are also transmitted to the monitor 466 to provide a visual display of the image. Specifically, the MR signals fill or form a k-space that is Fourier transformed to obtain a viewable image. The processed signals representative of the image are then transmitted to the monitor 466.

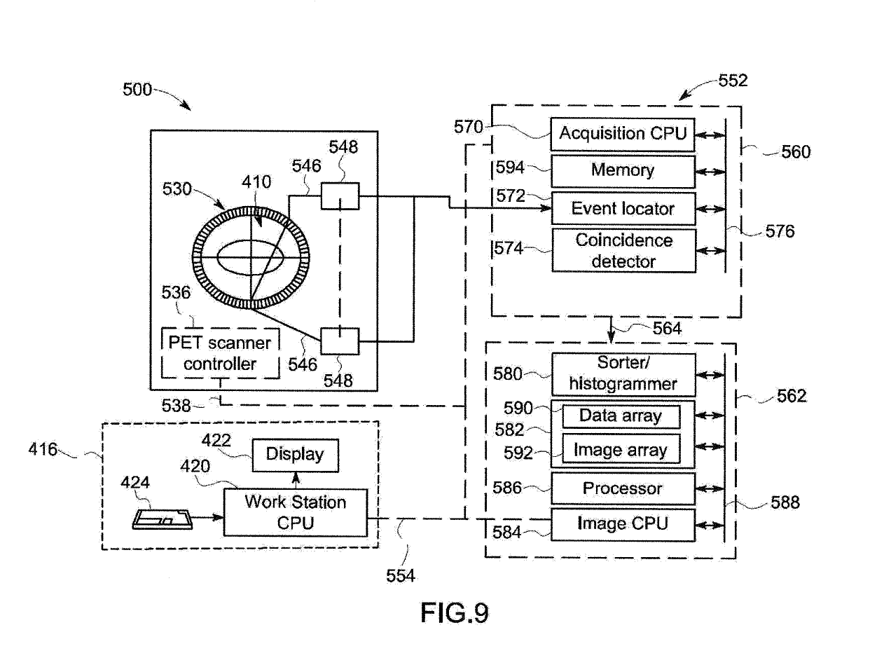

FIG. 9 is a diagram of an exemplary PET imaging system 500 that may form one of the modalities of the multi-modality imaging system 400 described above. The PET imaging system 500 includes a detector ring assembly 530 including a plurality of detector scintillators. The detector ring assembly 530 includes the central opening 410, in which an object or patient, such as the patient 405 may be positioned, using, for example, the motorized table 412 (not shown in FIG. 7). The scanning operation is controlled from the operator workstation 416 through a PET scanner controller 536. A communication link 538 may be hardwired between the PET scanner controller 536 and the workstation 416. Optionally, the communication link 538 may be a wireless communication link that enables information to be transmitted to or from the workstation 416 to the PET scanner controller 536 wirelessly. In the exemplary embodiment, the workstation 416 controls real-time operation of the PET imaging system 500. The workstation 416 is also programmed to perform medical image diagnostic acquisition and reconstruction processes described herein. The operator workstation 416 includes the central processing unit (CPU) or computer 420, the display 422 and the input device 424. As used herein, the term “computer” may include any processor-based or microprocessor-based system configured to execute the methods described herein.

The methods described herein may be implemented as a set of instructions that include various commands that instruct the computer or processor 420 as a processing machine to perform specific operations such as the methods and processes of the various embodiments described herein.

During operation of the exemplary detector 530, when a photon collides with a scintillator on the detector ring assembly 530, the absorption of the photon within the detector produces scintillation photons within the scintillator. The scintillator produces an analog signal that is transmitted on a communication link 546 when a scintillation event occurs. A set of acquisition circuits 548 is provided to receive these analog signals. The acquisition circuits 548 produce digital signals indicating the 3-dimensional (3D) location and total energy of each event. The acquisition circuits 548 also produce an event detection pulse, which indicates the time or moment the scintillation event occurred.

The digital signals are transmitted through a communication link, for example, a cable, to a data acquisition controller 552 that communicates with the workstation 416 and the PET scanner controller 536 via a communication link 554. In one embodiment, the data acquisition controller 552 includes a data acquisition processor 560 and an image reconstruction processor 562 that are interconnected via a communication link 564. During operation, the acquisition circuits 548 transmit the digital signals to the data acquisition processor 560. The data acquisition processor 560 then performs various image enhancing techniques on the digital signals and transmits the enhanced or corrected digital signals to the image reconstruction processor 562 as discussed in more detail below.

In the exemplary embodiment, the data acquisition processor 560 includes at least an acquisition CPU or computer 570. The data acquisition processor 560 also includes an event locator circuit 572 and a coincidence detector 574. The acquisition CPU 570 controls communications on a back-plane bus 576 and on the communication link 564. During operation, the data acquisition processor 560 periodically samples the digital signals produced by the acquisition circuits 548. The digital signals produced by the acquisition circuits 548 are transmitted to the event locator circuit 572. The event locator circuit 572 processes the information to identify each valid event and provide a set of digital numbers or values indicative of the identified event. For example, this information indicates when the event took place and the position of the scintillator that detected the event. The events are also counted to form a record of the single channel events recorded by each detector element. An event data packet is communicated to the coincidence detector 574 through the back-plane bus 576.

The coincidence detector 574 receives the event data packets from the event locator circuit 572 and determines if any two of the detected events are in coincidence. Coincident event pairs are located and recorded as a coincidence data packets by the coincidence detector 574. The output from the coincidence detector 574 is referred to herein as image data. In one embodiment, the image data may be stored in a memory device that is located in the data acquisition processor 560. Optionally, the image data may be stored in the workstation 416.

The image data subset is then transmitted to a sorter/histogrammer 580 to generate a data structure known as a histogram. The image reconstruction processor 562 also includes a memory module 582, an image CPU 584, an array processor 586, and a communication bus 588. During operation, the sorter/histogrammer 580 performs the motion related histogramming described above to generate the events listed in the image data into 3D data. This 3D data, or sinograms, is organized in one exemplary embodiment as a data array 590. The data array 590 is stored in the memory module 582. The communication bus 588 is linked to the communication link 576 through the image CPU 584. The image CPU 584 controls communication through communication bus 588. The array processor 586 is also connected to the communication bus 588. The array processor 586 receives the data array 590 as an input and reconstructs images in the form of image arrays 592. Resulting image arrays 592 are then stored in the memory module 582. The images stored in the image array 592 are communicated by the image CPU 584 to the operator workstation 416. In the illustrated embodiment, the PET imaging system 500 also includes a memory 594 that may be utilized to store a set of instructions to implement the various methods described herein.

The various embodiments and/or components, for example, the modules, or components and controllers therein, such as of the imaging system 400, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as an optical disk drive, solid state disk drive (e.g., flash RAM), and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” may include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

What is claimed is:

1. A method for performing an examination of a patient using a dual-modality imaging system, the method comprising:
accessing a list that includes a magnetic resonance imaging (MRI) examination protocol to be used to perform a predetermined MRI examination, the MRI examination protocol including a plurality of MRI protocol actions;
receiving a user input selecting at least one of the MRI protocol actions;
scanning the patient using the MRI examination protocol;
scanning the patient using a positron emission tomography (PET) imaging system to acquire PET data; and
marking the PET data, using a first visual indication, to indicate the selected MRI protocol action.

2. The method of claim 1, further comprising performing the MRI imaging system scan concurrently with the PET imaging system scan.

3. The method of claim 1, further comprising:
selecting an MRI system examination to be performed on a patient to acquire MRI data of the patient; and
selecting at least one MRI protocol action based on the selected MRI examination.

4. The method of claim 1, wherein the MRI protocol actions include at least one of activating an x-gradient coil, activating a y-gradient coil, activating a z-gradient coil or activating an RF coil.

5. The method of claim 1, further comprising:
generating a graphical illustration of the PET data; and
displaying a visual indication on the graphical illustration to indicate the selected MRI protocol action.

6. The method of claim 1, further comprising:
sorting the PET data based on the marking; and
reconstructing a PET image using the sorted PET data.

7. The method of claim 1, further comprising:
marking the PET data to indicate the selected MRI protocol action using a second different visual indication;
sorting the PET data based on the first and second visual indications; and
reconstructing a PET image using the sorted PET data.

8. The method of claim 1, further comprising:
removing the marked portion of the PET data to generate second set of PET data;
weighting the second set of PET data; and
reconstructing an image of a patient using the weighted PET data.

9. A multi-modality imaging system comprising:
a magnetic resonance imaging (MRI) system;
a positron emission tomography (PET) imaging system; and
a computer coupled to the MRI system and the PET system, the computer configured to:
access a list that includes a magnetic resonance imaging (MRI) examination protocol to be used to perform a predetermined MRI examination, the examination protocol including a plurality of MRI protocol actions;
receive a user input selecting at least one of the MRI protocol actions;
scan the patient using the MRI examination protocol;
scan the patient using a positron emission tomography (PET) imaging system to acquire PET data; and
mark the PET data to indicate the selected MRI protocol action.

10. The multi-modality imaging system of claim 9, wherein the computer is further configured to perform the MRI imaging system scan concurrently with the PET imaging system scan.

11. The multi-modality imaging system of claim 9, wherein the computer is further configured to:
receive an input selecting an MRI system examination to be performed on a patient to acquire MRI data of the patient; and
receive an input selecting at least one MRI protocol action based on the selected MRI examination.

12. The multi-modality imaging system of claim 9, wherein the MRI protocol actions include at least one of activating an x-gradient coil, activating a y-gradient coil, activating a z-gradient coil or activating an RF coil.

13. The multi-modality imaging system of claim 9, wherein the computer is further configured to:
generate a graphical illustration of the PET data; and
display a visual indication on the graphical illustration to indicate the selected MRI protocol action.

14. The multi-modality imaging system of claim 9, wherein the computer is further configured to:
sort the PET data based on the marking; and
reconstruct a PET image using the sorted PET data.

15. The multi-modality imaging system of claim 9, wherein the computer is further configured to:
mark the PET data to indicate the selected MRI protocol action using a second different visual indication;
sort the PET data based on the first and second visual indications; and
reconstruct a PET image using the sorted PET data.

16. The multi-modality imaging system of claim 9, wherein the computer is further configured to:
remove the marked portion of the PET data to generate second set of PET data;
weight the second set of PET data; and
reconstruct an image of a patient using the weighted PET data.

17. A non-transitory computer readable medium encoded with a program programmed to instruct a computer to:
access a list that includes a magnetic resonance imaging (MRI) examination protocol to be used to perform a predetermined MRI examination, the examination protocol including a plurality of MRI protocol actions;
receive a user input selecting at least one of the MRI protocol actions;
scan the patient using the MRI examination protocol;
scan the patient using a positron emission tomography (PET) imaging system to acquire PET data; and
mark the PET data to indicate the selected MRI protocol action.

18. The non-transitory computer readable medium of claim 17, further programmed to instruct the computer to perform the MRI imaging system scan concurrently with the PET imaging system scan.

19. The non-transitory computer readable medium of claim 17, further programmed to instruct the computer to:
receive an input selecting an MRI system examination to be performed on a patient to acquire MRI data of the patient; and
receive an input selecting at least one MRI protocol action based on the selected MRI examination.

20. The non-transitory computer readable medium of claim 17, further programmed to instruct the computer to:
generate a graphical illustration of the PET data; and
display a visual indication on the graphical illustration to indicate the selected MRI protocol action.
